(19) United States
(12) Patent Application Publication
(10) Pub. No.: US 2025/0134694 A1
(43) Pub. Date: May 1, 2025

(54) MEDICAL COMPONENT AND EXCREMENT STORAGE DEVICE

(71) Applicant: ALCARE CO., LTD., Tokyo (JP)

(72) Inventors: Yu KOBAYASHI, Tokyo (JP); Wataru OKUYAMA, Tokyo (JP)

(73) Assignee: ALCARE CO., LTD., Tokyo (JP)

(21) Appl. No.: 18/835,740

(22) PCT Filed: Mar. 29, 2023
(86) PCT No.: PCT/JP2023/012877

§ 371 Date: Aug. 5, 2024

(30) Foreign Application Priority Data
Mar. 31, 2022 (JP) 2022-059964

(51) Int. Cl. A61F 5/445

(52) U.S. Cl. CPC A61F 5/445

(57) ABSTRACT

[Problems] To provide a medical component that can reduce clogging of excrement while reducing leakage when closed, and improve comfort when worn.

[Solution] Provided is a medical component including: a mouth tool that is attached to a pouch in which excrement from a stoma is to be stored and has an opening having a tubular shape through which the excrement is discharged; and a cap having a core portion to be inserted into the opening, in which a cross-sectional shape of the opening is a substantially elliptical shape having a major diameter in a substantially horizontal direction relative to a body surface, a cross-sectional shape of the core portion is a substantially elliptical shape having a diameter equal to or smaller than the opening, one or more annular corrugations having a substantially elliptical shape are formed in an insertion direction on an outer wall of the core portion, the annular corrugation is formed such that a length of a width in the substantially vertical direction relative to the body surface is longer than a length of a width in a substantially horizontal direction relative to the body surface, and the cross-sectional shape of the opening has a smaller diameter than the cross-sectional shape of the core portion including the annular corrugation.

### TECHNICAL FIELD

The present invention relates to a medical component, and furthermore, relates to an excrement storage device for temporarily storing excrement, excreted liquid, gas, exudate, secreting fluid, and the like (hereinafter, collectively referred to as “excrement”, the same applies hereinafter) from the inside of a living body or the surface of a living body.

### BACKGROUND ART

When a person is unable to control the excretion of stool or urine by his or her own will or when a person has a disease of the digestive or urinary system organ, a surgical operation may be performed to guide the intestinal tract or ureter to the body surface, and a stoma may be constructed on the body surface. A person with a stoma needs to attach, to the stoma, a pouch that can temporarily store excrement from the stoma. In addition, a person with an opening or wound on the body surface due to another disease also needs to wear a pouch around the opening or wound to dispose of excrement discharged by drainage or the like.

For example, Patent Literature 1 discloses an ostomy device including a pouch that stores excrement, and a tubular mouth tool (opening) that is provided at the bottom of the pouch and can be closed with a cap.

### CITATION LIST

### Patent Literature

Patent Literature 1: Japanese Translation of PCT International Application Publication No. 2017-500985

### SUMMARY OF INVENTION

### Technical Problem

However, depending on the state of excrement, the mouth tool may become clogged with the excrement. There is a technique to increase the opening diameter of the mouth tool to reduce the clogging, but, in a state in which the device is worn on the body, the mouth tool having a large opening diameter becomes bulky under clothing and reduces comfort when the device is worn.

Thus, it is a principal object of the present invention to provide a medical component and an excrement storage device that can reduce clogging of excrement while reducing leakage when closed, and improve comfort when worn.

### Solution to Problem

The present invention provides a medical component including:
a mouth tool that is attached to a pouch in which excrement from a stoma is to be stored and has an opening having a tubular shape through which the excrement is discharged; and
a cap having a core portion to be inserted into the opening, in which
a cross-sectional shape of the opening is a substantially elliptical shape having a major diameter in a substantially horizontal direction relative to a body surface,
a cross-sectional shape of the core portion is a substantially elliptical shape having a diameter equal to or smaller than the opening,
one or more annular corrugations having a substantially elliptical shape are formed in an insertion direction on an outer wall of the core portion,
the annular corrugation is formed such that a length of a width in the substantially vertical direction relative to the body surface is longer than a length of a width in a substantially horizontal direction relative to the body surface, and
the cross-sectional shape of the opening has a smaller diameter than the cross-sectional shape of the core portion including the annular corrugation.

A minor diameter of the annular corrugation may be formed longer than a minor diameter of the opening.

A major diameter of the annular corrugation may be formed longer than the major diameter of the opening.

The mouth tool and the cap may be formed of the same resin material.

The mouth tool and the cap may be integrally formed.

In addition, the present invention provides an excrement storage device including: the medical component described above; and a pouch in which excrement from a stoma is to be stored, in which the pouch has a discharge port through which the excrement is discharged, and the medical component is attached to the discharge port.

### Advantageous Effects of Invention

According to the present invention, it is possible to reduce clogging of excrement while reducing leakage when closed, and improve comfort when worn. Note that the effects of the present invention are not necessarily limited to the effects described herein and may be any of the effects described in the present disclosure.

### BRIEF DESCRIPTION OF DRAWINGS

FIG. 1 is a perspective view showing a configuration example of a medical component according to an embodiment of the present invention.

FIG. 2 is a front view showing the configuration example of the medical component according to the embodiment of the present invention.

FIG. 3 is a bottom view showing the configuration example of the medical component according to the embodiment of the present invention.

FIG. 4 is a front view showing the configuration example of the medical component according to the embodiment of the present invention.

FIG. 5 is a front view showing a configuration example of an excrement storage device according to an embodiment of the present invention.

### DESCRIPTION OF EMBODIMENTS

Hereinbelow, preferred embodiments for carrying out the present invention will be described with reference to the drawings. Note that the embodiments described below show examples of the representative embodiment of the present invention and do not limit the scope of the present invention. In addition, the present invention can combine any of the following embodiments and modifications thereof.

In the following description of the embodiments, the configuration may be described using terms with “substantially” such as “substantially parallel” and “substantially perpendicular”. For example, “substantially parallel” means not only being perfectly parallel, but also being substantially parallel, that is, including a state deviated from a perfectly parallel state by, for example, approximately a few percent (e.g., a deviation within 0.5, 1, 2, or 3% or less). The same applies to other terms with “substantially”. In addition, each drawing is a schematic diagram and is not necessarily strictly illustrated.

Unless otherwise noted, in the drawings, “up” means the upper direction or the upper side in the drawings, “down” means the lower direction or the lower side in the drawings, “left” means the left direction or the left side in the drawings, and “right” means the right direction or the right side in the drawings. In addition, in the drawings, identical or equivalent elements or members are designated by the same reference signs, and redundant description will be omitted.

The present invention can provide a medical component including: a mouth tool that is attached to a pouch in which excrement from a stoma is to be stored and has an opening having a tubular shape through which the excrement is discharged; and a cap having a core portion to be inserted into the opening, in which a cross-sectional shape of the opening is a substantially elliptical shape having a major diameter in a substantially horizontal direction relative to a body surface, a cross-sectional shape of the core portion is a substantially elliptical shape having a diameter equal to or smaller than the opening, one or more annular corrugations having a substantially elliptical shape are formed in an insertion direction on an outer wall of the core portion, the annular corrugation is formed such that a length of a width in the substantially vertical direction relative to the body surface is longer than a length of a width in a substantially horizontal direction relative to the body surface, and the cross-sectional shape of the opening has a smaller diameter than the cross-sectional shape of the core portion including the annular corrugation.

The medical component is, for example, a medical component used in an ostomy device pouch, a drainage pouch, a defecation bag, a urine storage bag, and a urine collection bag. One or two or more kinds can be selected from these.

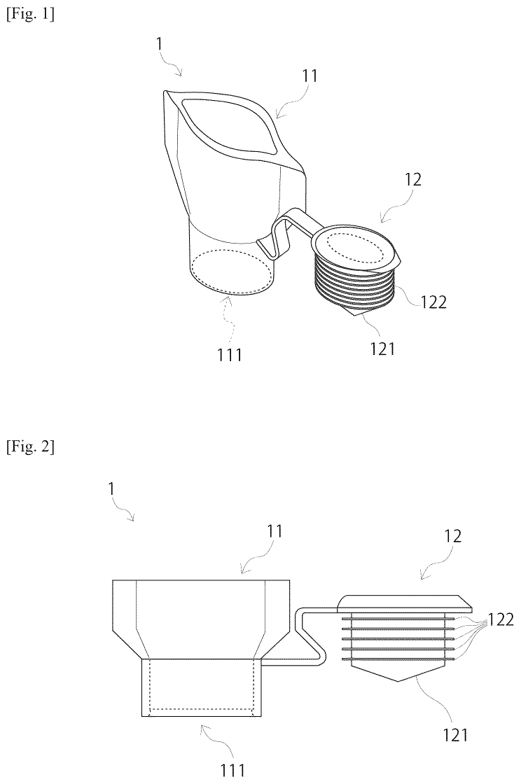

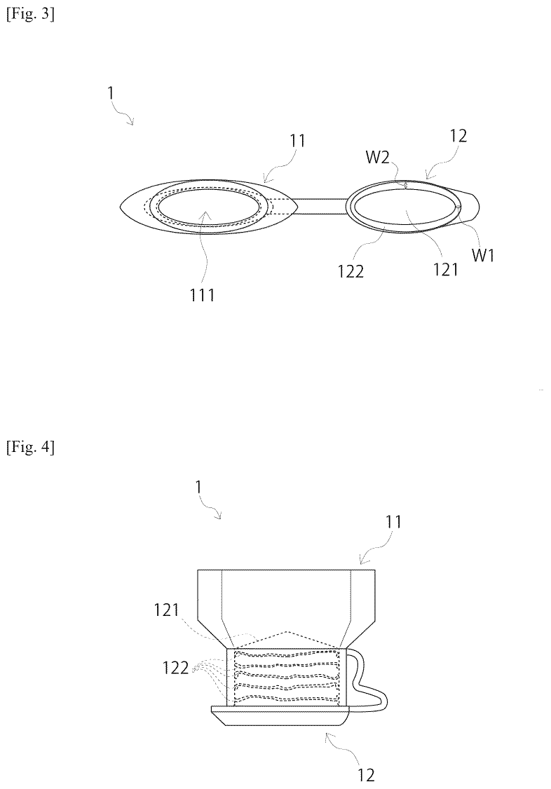

A configuration example of the medical component according to an embodiment of the present invention will be described with reference to FIGS. 1 to 3. FIG. 1 is a perspective view showing the configuration example of a medical component 1 according to the embodiment of the present invention. FIG. 2 is a front view showing the configuration example of the medical component 1 according to the embodiment of the present invention. FIG. 3 is a bottom view showing the configuration example of the medical component 1 according to the embodiment of the present invention.

As shown in FIG. 2, the medical component 1 of according to the embodiment of the present invention includes a mouth tool 11 that is attached to a pouch in which excrement from a stoma is to be stored and has an opening 111 having a tubular shape through which the excrement is discharged, and a cap 12 having a core portion 121 to be inserted into the opening 111. The tubular shape includes, for example, a hollow substantially cylindrical shape. Examples of the hollow substantially cylindrical shape include a hollow substantially perfect cylindrical shape, a hollow substantially elliptical cylindrical shape, and tapered shapes of these (such as a hollow inverted substantially elliptical truncated cone and a hollow inverted substantially circular truncated cone). Note that, for “substantially elliptical” of the substantially elliptical cylinder and the inverted substantially elliptical truncated cone, the description of “substantially elliptical shape” described below can be employed as appropriate. One or two or more kinds can be selected from these.

As shown in FIG. 3, the cross-sectional shape of the opening 111 is a substantially elliptical shape having a major diameter in a substantially horizontal direction relative to the body surface. This makes it possible to reduce discomfort caused by the device becoming bulky under clothing when the device is worn on the body. The cross-sectional shape of the opening 111 is preferably the cross-sectional shape of a hollow portion of the tubular shape of the opening 111. The hollow portion may be a space portion on the inner wall side of the opening 111 or inside the opening 111. The substantially elliptical shape may include a polygon with rounded corners in addition to an elliptical shape. The polygon with rounded corners includes, for example, a polygon that is vertically and/or horizontally symmetrical and has rounded corners, and more specific examples thereof include a rounded corner quadrangle of a horizontally elongated type and a rounded corner polygon of a horizontally elongated type close to an elliptical shape. In this case, the length of the width in the longitudinal direction (the maximum length in the longitudinal direction) of the rounded corner polygon desirably corresponds to the major diameter of the elliptical shape, and the length of the width in the short direction (the maximum length in the short direction) desirably corresponds to the minor diameter of the elliptical shape. Examples of the rounded corner quadrangle include a rounded corner rhombus, a rounded corner parallelogram, and a rounded corner rectangle. One or two or more kinds may be selected from these as appropriate.

In addition, even when the opening 111 gets clogged with excrement, the width in the short direction (a substantially vertical direction relative to the body surface) of the opening 111 is increased by the wearer applying a force to an end in the longitudinal direction (the substantially horizontal direction relative to the body surface) of the cross section of the opening 111, thereby making it possible to facilitate discharge of the excrement and clear the clogging.

Thus, the medical component 1 according to the present embodiment can reduce clogging of excrement while reducing leakage when closed, and improve comfort when worn.

The cross-sectional shape of the core portion 121 is a substantially circular shape having a diameter equal to or smaller than that of the opening 111, and preferably a substantially elliptical shape that extends along the inner wall of the opening 111 when inserted into the opening 111. The major diameter and the minor diameter of the core portion 121 may be equal to the major diameter and the minor diameter of the opening 111, respectively. The cross-sectional shape of the core portion 121 may be a substantially elliptical shape having a diameter (major and minor diameters) equal to or smaller than the diameter (major and minor diameters of the inner diameter) of the cross-sectional shape of the hollow portion of the opening 111. The size of the cross-sectional shape of the core portion 121 is desirably set, also taking into consideration one or more annular corrugations on the outer wall of the core portion. In addition, although not shown in the drawings, a cavity may be formed in a shaft of the core portion 121 for weight reduction.

As shown in FIG. 1, one or more annular corrugations 122 that have elasticity and have a substantially elliptical shape are formed in an insertion direction on the outer wall of the core portion 121. This annular corrugation 122 can reduce leakage of excrement through a gap between the opening 111 and the cap 12 by coming into contact with the inner wall of the opening 111 and filling the gap between the core portion 121 and the inner wall of the opening 111 when the opening 111 is closed with the cap 12.

As shown in FIG. 3, the annular corrugation 122 is formed such that the length of a width W2 in the short direction of the core portion 121 (the substantially vertical direction relative to the body surface) is longer than the length of a width W1 in the longitudinal direction of the core portion 121 (the substantially horizontal direction relative to the body surface). This makes it possible to make a gap less likely to be formed between the inner wall of the opening 111 and the annular corrugation 122 even when, for example, an external force is applied to the opening 111 and the cross-sectional shape of the opening 111 is deformed into a substantially perfect circular shape. As a result, leakage of excrement through the gap can be reduced.

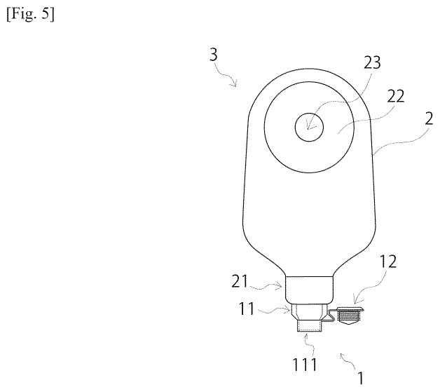

The cross-sectional shape of the opening 111 preferably has a diameter equal to or smaller than that of the “cross-sectional shape of the core portion 121”. This will be described with reference to FIG. 4. FIG. 4 is a front view showing the configuration example of the medical component 1 according to the embodiment of the present invention, and shows a state in which the opening 111 is closed with the cap 12. Although, in FIG. 4, the cap 12 has a projecting portion that has a substantially elliptical cone shape and projects toward the pouch 2 relative to the inner wall of the opening 111, the cap 12 may be one without the projecting portion (e.g., one with a flat tip).

In the present embodiment, the cross-sectional shape of the opening 111 preferably has a smaller diameter than the “cross-sectional shape of the core portion 121 including the annular corrugation 122”. Thus, as shown in FIG. 4, an end of the annular corrugation 122 is bent in contact with the inner wall of the opening 111. This makes it possible to reduce leakage of excrement. For example, even when an external force is applied to the opening 111 and the cross-sectional shape of the opening 111 is deformed, the annular corrugation 122 follows the deformation, thereby making it possible to make a gap, which causes leakage, less likely to be formed between the inner wall of the opening 111 and the annular corrugation 122.

More preferably, the minor diameter of the annular corrugation 122 is formed to be longer than the minor diameter of the opening 111. The major diameter of the annular corrugation 122 is formed to be longer than the major diameter of the opening 111. Accordingly, even when a strong external force is applied to the opening 111 and the opening 111 is deformed into a substantially perfect circular shape, it is possible to make a gap, which causes leakage, less likely to be formed between the inner wall of the opening 111 and the annular corrugation 122.

In addition, the core portion 121 preferably has a predetermined thickness and a predetermined strength. This makes it possible to prevent the cross-sectional shape of the opening 111 from being further deformed into a substantially elliptical shape having a minor diameter in the substantially horizontal direction relative to the body surface after being deformed from an elliptical shape having a major diameter in the substantially horizontal direction relative to the body surface into a substantially perfect circular shape, when an external force is applied to the opening 111.

The number of annular corrugations 122 may be one, but preferably more than one. In the present embodiment, one or more annular corrugations 122 are formed in the insertion direction. Forming one or more annular corrugations 122 makes it possible to further reduce leakage of excrement.

The annular corrugation 122 may have a predetermined elasticity. This makes it possible to open and close the cap 12 with a weak force.

The mouth tool 11 and the cap 12 may be made of the same resin material, and, for example, a flexible resin material is preferred. A connecting member (e.g., a substantially Z shape) that connects the mouth tool 11 and the cap 12 may be provided in the longitudinal direction of the mouth tool 11. The mouth tool 11 and the cap 12 may be integrally formed. This makes it possible to manufacture the medical component 1 at low cost. As an example of the resin material, an ultraviolet-curable resin, a thermoplastic elastomer resin, or the like can be used. Each part of the medical component can employ a flexible resin (molded article) as appropriate, and a flexible resin is preferably used for the mouth tool 11 and/or the cap 12.

The size of the medical component, and each of the parts, such as the opening 111, the mouth tool 11, the core portion 121, and the cap 12, can be appropriately set taking into consideration the excrement storage device to be used. As a preferred example, regarding the size of the cross-sectional shape (substantially elliptical shape) of the hollow portion of the opening 111, the length of the width in the longitudinal direction (the major diameter of the inner diameter) of this cross-sectional shape is preferably 5 to 150 mm and more preferably 15 to 40 mm, the length of the width in the short direction (the minor diameter of the inner diameter) of this cross-sectional shape is preferably 3 to 50 mm and more preferably 5 to 20 mm, and the length of the width in the short direction (the minor diameter of the inner diameter) relative to the length of the width in the longitudinal direction (the major diameter of the inner diameter) (100%) can be set to, for example, preferably 5 to 95% and more preferably 10 to 85%. Note that, in the present specification, the upper limit (equal to or less than) and the lower limit (equal to or more than) of each numerical value range can be combined in any manner as desired, and for example, in the above case, 5 to 50 mm in the major diameter, 3 to 20 mm in the minor diameter, and 5 to 85% or 10 to 95% in the ratio of the minor diameter to 100% of the major diameter.

### (Excrement Storage Device)

The present invention provides an excrement storage device including the medical component according to the other embodiment described above, and a pouch in which excrement from a stoma is to be stored, in which the pouch has a discharge port through which the excrement is discharged, and the medical component is attached to the discharge port.

Examples of the excrement storage device include an ostomy device, a drainage pouch, a defecation bag, a urine storage bag, and a urine collection bag, and one or two or more kinds can be selected from these.

The excrement storage device according to an embodiment of the present invention will be described with reference to FIG. 5. FIG. 5 is a front view showing a configuration example of an excrement storage device 3 according to the embodiment of the present invention.

The excrement storage device 3 shown in FIG. 5 is an ostomy device as an example. As shown in FIG. 5, the excrement storage device 3 according to the embodiment of the present invention includes the medical component 1, and a pouch 2 in which excrement from a stoma is to be stored. As shown in FIG. 4, excrement is stored in the pouch 2 by closing the opening 111 of an excretion port 21 with the cap 12. As shown in FIG. 5, by removing the cap 12 from the opening 111, the opening 111 is opened.

The pouch 2 is formed in a sealed bag shape using a plastic film. A faceplate 22 is provided at the upper part of the excrement storage device 3. The pouch 2 is attached to the stoma of a patient through an adhesive layer of the faceplate 22. Excrement is discharged into the pouch 2 through a hole 23 of the faceplate 22.

The pouch 2 has a discharge port 21 through which the excrement is discharged. The discharge port 21 is attached to the medical component 1 by heat welding or the like.

The excrement stored in the pouch 2 passes through the discharge port 21 and is discharged through the opening 111 of the mouth tool 11 included in the medical component 1.

Thus, the excrement storage device 3 can reduce clogging of excrement while reducing leakage when closed, and improve comfort when worn.

### Example

The effects of the present invention will be specifically described using an example. Note that the scope of the present invention is not limited to the example described in the present specification.

### <Experimental Method>

A 3D printer (J750 Digital Anatomy, manufactured by Stratasys Ltd.) was used to form the medical component. Agilus 30 manufactured by Stratasys Ltd. was used as the resin material to form the medical component. Note that, although this resin material is not the resin material actually used (e.g., an ultraviolet-curable resin or a thermoplastic elastomer resin), this resin material can obtain the same effect as the resin material actually used. Furthermore, the resin material actually used may obtain a better effect.

The design of the medical component will be described with reference to Table 1 below.

TABLE 1

| | Example | | Comparative Example | |
|---|---|---|---|---|
| Major diameter of opening | 35.8 | mm | 35.8 | mm |
| Minor diameter of opening | 12.8 | mm | 12.8 | mm |
| Major diameter of core portion | 35.8 | mm | 35.8 | mm |
| Minor diameter of core portion | 12.8 | mm | 12.8 | mm |
| Width of annular corrugation | 3.0 | mm | 0.5 | mm |
| (longitudinal direction) | | | | |
| Width of annular corrugation | 6.5 | mm | 0.5 | mm |
| (short direction) | | | | |

As shown in Table 1, in both the example and the comparative example, the major diameter and the minor diameter of the opening (inner diameter) of the mouth tool were 35.8 mm and 12.8 mm, respectively. The major diameter and the minor diameter of the cap (core portion) were the same as the major diameter and the minor diameter of the opening (inner diameter), respectively.

In the example, the width of the annular corrugation (the length from the outer circumference of the core portion of the cap to the tip of the annular corrugation, the same applies hereinafter) was 3.0 mm in the longitudinal direction and 6.5 mm in the short direction. The width in the short direction is formed longer than the width in the longitudinal direction.

On the other hand, in the comparative example, the width of the annular corrugation was 0.5 mm in both the longitudinal direction and the short direction. The width in the longitudinal direction and the width in the short direction are formed to be the same length.

Furthermore, in the example, when the opening (elliptical shape) is deformed into a substantially perfect circular shape, the minor diameter of the annular corrugation becomes longer than the minor diameter of the opening.

Next, with the opening closed with the cap (refer to FIG. 4), water was put into the mouth tool 11, and each of “A. Do nothing and allow it to stand”, “B. Apply finger pressure 10 times in the short direction”, and “C. Apply finger pressure 10 times in the longitudinal direction” was performed, and the degree of water leakage was checked.

### <Experimental Result>

An experimental result will be described with reference to Table 2 below.

TABLE 2

| | | | Comparative |
|---|---|---|---|
| | | Example | Example |
| A. | Do nothing and allow it | No leakage occurred | No leakage occurred |
| | to stand | | |
| B. | Apply finger pressure 10 | No leakage occurred | No leakage occurred |
| | times in short direction | | |
| C. | Apply finger pressure 10 | No leakage occurred | Leakage occurred |
| | times in longitudinal | | |
| | direction | | |

As shown in Table 2, no water leakage occurred in both the cases of “A. Do nothing and allow it to stand” and “B. Apply finger pressure 10 times in the short direction”.

However, when “C. Apply finger pressure 10 times in the longitudinal direction” was performed, no water leakage occurred in the example, but water leakage occurred in the comparative example. This is because, in the comparative example, the cross-sectional shape of the opening was deformed into a substantially perfect circular shape due to an external force applied to the opening, which formed a gap between the inner wall of the opening and the annular corrugation, and water leaked through this gap.

In this manner, the medical component according to the present invention can reduce clogging of excrement while reducing leakage when closed, and improve comfort when worn.

In addition, the present invention can also be configured as follows.

[1]

A medical component including:
a mouth tool that is attached to a pouch in which excrement from a stoma is to be stored and has an opening having a tubular shape through which the excrement is discharged; and
a cap having a core portion to be inserted into the opening, in which
a cross-sectional shape of the opening is a substantially elliptical shape having a major diameter in a substantially horizontal direction relative to a body surface,
a cross-sectional shape of the core portion is a substantially elliptical shape having a diameter equal to or smaller than the opening,
one or more annular corrugations having a substantially elliptical shape are formed in an insertion direction on an outer wall of the core portion,
the annular corrugation is formed such that a length of a width in the substantially vertical direction relative to the body surface is longer than a length of a width in a substantially horizontal direction relative to the body surface, and
the cross-sectional shape of the opening has a smaller diameter than the cross-sectional shape of the core portion including the annular corrugation.
   [2]

The medical component according to [1], in which a minor diameter of the annular corrugation is formed longer than a minor diameter of the opening.

[3]

The medical component according to [1] or [2], in which a major diameter of the annular corrugation is formed longer than the major diameter of the opening.

[4]

The medical component according to any one of [1] to [3], in which the mouth tool and the cap are formed of the same resin material.

[5]

The medical component according to any one of [1] to [4], in which the mouth tool and the cap are integrally formed.

[6]

An excrement storage device including:
the medical component according to any one of [1] to [5]; and
a pouch in which excrement from a stoma is to be stored, in which the pouch has a discharge port through which the excrement is discharged, and the medical component is attached to the discharge port.

### REFERENCE SIGNS LIST

1 medical component
11 mouth tool
111 opening
12 cap
121 core portion
122 annular corrugation
2 pouch
21 discharge port
22 faceplate
23 hole
3 excrement storage device

1. A medical component comprising:
a mouth tool that is attached to a pouch in which excrement from a stoma is to be stored and has an opening having a tubular shape through which the excrement is discharged; and
a cap having a core portion to be inserted into the opening, wherein
a cross-sectional shape of the opening is a substantially elliptical shape having a major diameter in a substantially horizontal direction relative to a body surface,
a cross-sectional shape of the core portion is a substantially elliptical shape having a diameter equal to or smaller than the opening,
one or more annular corrugations having a substantially elliptical shape are formed in an insertion direction on an outer wall of the core portion,
the annular corrugation is formed such that a length of a width in the substantially vertical direction relative to the body surface is longer than a length of a width in a substantially horizontal direction relative to the body surface, and
the cross-sectional shape of the opening has a smaller diameter than the cross-sectional shape of the core portion including the annular corrugation.

2. The medical component according to claim 1, wherein a minor diameter of the annular corrugation is formed longer than a minor diameter of the opening.

3. The medical component according to claim 1,
wherein a major diameter of the annular corrugation is formed longer than the major diameter of the opening.

4. The medical component according to claim 1, wherein the mouth tool and the cap are formed of the same resin material.

5. The medical component according to claim 1, wherein the mouth tool and the cap are integrally formed.

6. An excrement storage device comprising:
the medical component according to claim 1; and
a pouch in which excrement from a stoma is to be stored, wherein
the pouch has a discharge port through which the excrement is discharged, and
the medical component is attached to the discharge port.

7. The medical component according to claim 2, wherein a major diameter of the annular corrugation is formed longer than the major diameter of the opening.

8. The medical component according to claim 2, wherein the mouth tool and the cap are formed of the same resin material.

9. The medical component according to claim 2, wherein the mouth tool and the cap are integrally formed.

10. The medical component according to claim 3, wherein the mouth tool and the cap are formed of the same resin material.

11. The medical component according to claim 3, wherein the mouth tool and the cap are integrally formed.

12. The medical component according to claim 4, wherein the mouth tool and the cap are integrally formed.
